(19) United States
(12) Patent Application Publication
(10) Pub. No.: US 2008/0064642 A1
(43) Pub. Date: Mar. 13, 2008

(54) Peptidic And Peptidoid Bradykinin B1 Receptor Antagonists And Uses Thereof

(75) Inventors: Brigitte Guerin, Saint - Elie - d'Orford (CA); Bruno Battistini, Orford (CA); Fernand Gobeil, Sherbrooke (CA); Francois Nantel, Sherbrooke (CA); Witold Neugebauer, Ottawa (CA); Gerard Plante, Sherbrooke (CA); Domenico Regoli, Magog (CA); Pierre Sirois, Orford (CA)

Correspondence Address: DAVID S. RESNICK, 100 SUMMER STREET, NIXON PEABODY LLP, BOSTON, MA, 02110-2131, US

(73) Assignee: UNIVERSITE DE SHERBROOKE, Sherbrooke, Quebec (CA)

(21) Appl. No.: 11/660,436

(22) PCT Filed: Aug. 19, 2005
(86) PCT No.: PCT/CA05/01268

§ 371 Date: Aug. 20, 2007

Related U.S. Application Data
(60) Provisional application No. 60/602,626, filed on Aug. 19, 2004.

(51) Int. Cl. A61K 38/08; A61P 25/00; C07K 7/06

(52) U.S. Cl. 514/16; 530/328; 530/329

(57) ABSTRACT

The present invention provides for new peptidic and peptidoid Bradykinin B₁ receptor antagonists of formula (1) having good to excellent affinities and selectivity for the BKB₁ receptor, and increased resistance to enzymatic degradation, superior pharmacokinetic properties, both in vitro and in vivo, with capability to significantly prevent and treat conditions wherein BKB₁Rs are induced and over-expressed.

### TECHNICAL FIELD

The present invention relates to new biologically active peptidic or peptidoid derivatives, selective and potent antagonists to the bradykinin (BK) B₁ receptors (BKB₁Rs) and their uses, for preventing and treating conditions wherein BKB₁Rs are induced and over-expressed.

### BACKGROUND OF THE INVENTION

Bradykinin, an autacoid nonapeptide, plays an important role in normal physiological processes in healthy individuals, as well as in acute and chronic pathological conditions (Farmer S. G., 1997, The Kinin System, Academic Press, San Diego, 349 p.). The kallikrein-kinin system is composed of two major proteolytic systems (in plasma and tissues) that are responsible for the liberation of BK and kallidin (LysBK), which can be further converted to BK by aminopeptidases (Skidgel, 1992, J Cardiovasc Pharmacol, 20: S4-9). The Half life of kinins is estimated to be less than 30 sec in human. Nevertheless, once synthesized and released, BK can bind to two receptor subtypes, called BKB₁ and BKB₂ (referred to herein as BKB₁R and BKB₂R), (Regoli and Barabé, 1980, Pharmacol Rev, 32: 1-46). BKB₂R is expressed in normal tissues and in a variety of cells as endothelia, smooth muscles, epithelia and white blood cells. BKB₂R mediates smooth muscle contraction and the release of autacoids, particularly from the endothelium. This function provides the basic mechanism of peripheral vasodilatation which is responsible for a large part of the in vivo hypotensive effect of BK through BKB₂Rs (Resende et al., 1998, Braz J Med Biol Res, 31: 1229-1235). BKB₁R is induced by various pro-inflammatory stimuli (lipopolysaccharide, cytokines) in several cell types including endothelial, smooth muscle, blood cells, and neurons (Marceau et al. 1998, Pharmacol Rev, 50: 357-386).

Inducible BKB₁Rs are involved in various types of pain and inflammatory syndromes (Marceau et al. vide supra; Couture et al. 2001, Eur J Pharmacol, 429: 161-176), in diabetes (Zuccollo. et al. 1996, Can J Physiol Pharmacol, 74: 586-589) and related complications (Simard et al., 2002, Can J Physiol Pharmacol, 80: 1203-1207; Qabra and Sirois, 2003, Peptides, 24: 1131-1139), in allergies and asthma (Perron et al., 1999, Eur J Pharmacol, 376: 83-89; Ozturk et al. 2001, Curr Pharma Des, 7: 135-161), in arthritis (Davis and Perkins, 1994, Neuropharmacol, 33: 127-133), in diabetic and other types of vasculopathies, in vascular and non-vascular remodeling (Spillmann et al., 2002, Int Immunopharmacol, 2: 1823-1832), in fibrosis, in angiogenesis (Marceau et al. vide supra; Emanueli et al., 2002, Circ 105: 360-366), in proliferation, and cancers (Ishihara et al., 2002, Int Immunopharmacol, 2: 499-509). Dray and Perkins (1993, Trends Neurosci, 16: 99-104) have reviewed the possible implication of BKB₁Rs in various inflammatory conditions, tissue reactions to noxious stimuli and hyperalgesia, first alongside the acute phase, but particularly the chronic phases of these disturbances.

Peptidic BKB₁ receptor antagonists have been first developed in the late seventies (Regoli et al. 1977, Can J Physiol Pharmacol, 55: 855-867; Regoli and Barabé, vide supra). In more recent years (1997-now), international efforts for developing potent and selective BKB₁R antagonists, either peptidic and non-peptidic, have been reported (WO 97/09346, WO 97/25315, WO 00/75107, WO 01/05783, WO 02/099388, WO 03/106428, WO 03/066577).

Despite these BKB₁R antagonists cited in the prior art, it would be highly desirable to provide peptidic BKB₁R antagonists that present very good to high potency, affinity, selectivity and specificity for the BKB₁R, and that are resistant to various proteolytic enzyme degradation (Neugebauer et al. 2002, Can J Physiol Pharmacol, 80: 287-292), with chemical features that favor their absorption and general distribution (pharmacokinetic) in the body, excluding passage through the hemato-encephalic barrier, in order to optimize potency and duration of action in vivo, with a minimal toxicological/toxicokinetic profile. Furthermore, it would be interesting to investigate the inducible over-expressed BKB₁R subtype as a potential therapeutic target for a drug-preventive and curative approach to pain and inflammatory syndromes, as well as for cardiovascular, pulmonary, renal, diabetic and non-diabetic vasculopathies related to microvascular leakage, pro-inflammatory cell infiltration and activation in organs and tissues.

### SUMMARY OF THE INVENTION

One aim is to provide new peptidic BKB₁R antagonists that present potency, affinity, selectivity and specificity for the BKB₁R. In accordance with the present invention, there is provided a new biologically active peptidic or peptidoid derivatives of general formula (1), which act as potent, selective and specific antagonists of BKB₁R:
R-(Aaa⁰-Arg¹-Aaa²-Aaa³-Aaa⁴-Aaa⁵-Ser⁶-D-βNal⁷-Aaa⁸-OH)ₓ (1);
R is an acetyl group, or a hydrophobic extension;
Aaa⁰ is Orn, Lys, a basic amino acid or a salt of one of Orn, Lys or a basic amino acid, this basic amino acid can be either Arg or Cit;
Aaa² is Oic, Pro or a Pro mimic amino acid such as Hyp or α(Me)Pro, or a Pro mimic derivative, and preferably Oic;
Aaa³ is Pro, or a Pro mimic amino acid such as Hyp, Oic or α(Me)Pro, or a Pro mimic derivative, and preferably Pro;
Aaa⁴ is Gly, or H₂N—(CH₂)₂, or Aib, and preferably Gly;
Aaa⁵ is α(Me)Phe, Phe, D-α(Me)Phe, D-Phe, Cha, Cpa, Phg, Atc, Thi, Iglb, Aic, Chg, Cpg, Aib, AC6, AC5, AC4, AC3, and preferably α(Me)Phe, Cha, Thi, Phg, and Aic;
Aaa⁸ is Ile, or Leu, or Nle, and preferably Ile; and
x is 1 or 2.

Pro mimic derivatives or Pro mimic amino acid can be for examples in the various embodiment of the invention either Hyp, Oic or α(Me)Pro.

Alternatively, the residues -Aaa²-Aaa³-Aaa⁴- may be linked together to form a group selected from aliphatic, aromatic-aliphatic, heterocyclic or alicyclic group. Furthermore, -Aaa²-Aaa³-Aaa⁴-Aaa⁵- may be linked together to form a group selected from aliphatic, heterocyclic or alicyclic group.

The hydrophobic extension designated by R in formula (1) is an aliphatic, aromatic-aliphatic acylating group or an aliphatic, an aromatic-aliphatic group. The compound of formula (1) is in free base form or in salt form with an acid or a base.

The bradykinin peptidic or peptidoid antagonists of the present invention may be illustrated by the following. Some of these antagonists are characterized by an extended hydrophobic side chain which has been found to improve antagonist potency. The residue alignment in a particular row does not imply nor limit to a given peptide sequence.

| R | (Aaa⁰ | Arg¹ | Aaa² | Aaa³ | Aaa⁴ | Aaa⁵ | Ser⁶ | D-βNal⁷ | Ile⁸ | OH)₁ |
|---|---|---|---|---|---|---|---|---|---|---|
| n-C₃H₇CO | Orn | | Oic | Pro | Gly | α(Me)Phe | | | | |
| n-C₅H₁₁CO | Lys | | Pro | Hyp | NH—CH₂—CH₂ | Phe | | | | |

| | | |
|---|---|---|
| n-C₇H₁₅CO | NH—CH₂—C₆H₄—CH₂—CO | D-α(Me)Phe |
| n-C₉H₁₉CO | NH—C₆H₄—CH₂—CO | D-Phe |
| n-C₁₁H₂₃CO | NH—CH₂-biphenyl-CO | Cha |
| pMeO—C₆H₄—COC₄H₈CO | NH—(CH₂)₇—CO | Cpa |
| pMeO—C₆H₄—COC₆H₁₂CO | | Phg |
| pMeO—C₆H₄—CO—C₄H₈CO | | Atc |
| pMeO—C₆H₄—COC₆H₁₂CO | | Thi |
| CH₃ | | Iglb |
| C₂H₅ | | Aic |
| n-C₃H₇ | | Chg |
| n-C₄H₉ | | Cpg |
| n-C₆H₁₃ | | Aib |
| n-C₈H₁₇ | | AC6 |
| n-C₁₀H₂₁ | | AC5 |
| n-C₁₂H₂₅ | | AC4 |
| Ac | | AC3 |

NH—(CH₂)₁₀—CO
amino-ethyl-2,4-dioxo-3,4-dihydro-2H-
quinazolin-1-yl
piperidin-4-yl-2-oxo-2,3-dihydro-
benzoimidazol-1-yl
4-oxo-1-phenyl-1,3,8-triazaspiro
[4.5]dec-3-yl
4-oxo-1-cyclohexyl-1,3,8-triazaspiro
[4.5]dec-3-yl

In addition, the present invention provides homo-dimerized BKB₁ receptor antagonist described by the following formula. Homodimers are formed by linking two molecules of nonpeptides on resin by means of di-functionalized spacers (diacyl chloride or diol through a Mitsunobu alkylation; Wisniewski 2002, PEPTIDES Proceedings of the 27^{th} European Peptide Symposium, Sorrento, 322-323) via the N-terminal amino groups.

| R | (Aaa⁰ | Arg¹ | Aaa² | Aaa³ | Aaa⁴ | Aaa⁵ | Ser⁶ | D-βNal⁷ | Ile⁸ | OH)₂ |
|---|---|---|---|---|---|---|---|---|---|---|
| [C_{b}H_{2b}—CO]₂ | Orn | | Oic | Pro | Gly | α(Me)Phe | | | | |
| where b is an | | | | | | | | | | |
| integer from 2 to 5 | | | | | | | | | | |
| [n-C_{c}H_{2c}]₂ | Lys | | Pro | | | Phe | | | | |
| where c is an | | | | | | | | | | |
| integer from 3 to 6 | | | | | | | | | | |

According to the above formula, compound (1) may exist in free base form or in salt form with acids or bases in free base. The salts are generally prepared with pharmaceutically acceptable acids or bases. However, salts of other acids or bases which are useful for the purification or isolation of the compound of formula (1) also form part of the invention.

Further in accordance with the present invention, there is provided a method for treating a patient affected by a condition wherein the BKB₁R subtype is induced, over-expressed and subsequently mediate a response. Such conditions could result and are present alongside various types of acute and chronic phases of inflammation and injury (brain-neurogenic, vascular, pulmonary, respiratory, renal, bowel, skin, arthritis), cough, pain (dental, skin, bone, cancer, perioperative), cell migration, remodeling, proliferation, fibrosis, allergy (asthma, rhinitis, chronic obstructive pulmonary disease), cancer, coronary/cardiovascular diseases (vasospasm, myocardial ischemia, heart failure, hypertensions, stroke), diabetes, complications related to diabetes (vascular complications, nephropathy, microangiopathy, retinopathy, neuropathy), and vasculopathies-related to microvascular leakage.

Therefore, in accordance with the present invention, there is provided a method for treating cancer, malignant disease or related condition, selected from the group consisting of breast cancer, ovarian cancer, cervical carcinoma, endometrial carcinoma, choriocarcinoma, soft tissue sarcomas, osteosarcomas, rhabdomyosarcomas, leiomyomas, leiomyosarcomas, head and neck cancers, lung and bronchogenic carcinomas, brain tumors, neuroblastomas, esophageal cancer, colorectal adenocarcinomas, bladder cancer, urothelial cancers, leukemia, lymphoma, malignant melanomas, oral squamous carcinoma, hepatoblastoma, glioblastoma, astrocytoma, medulloblastoma, Ewing's sarcoma, lipoma, liposarcoma, malignant fibroblast histoma, malignant Schwannoma, testicular cancers, thyroid cancers, Wilms' tumor, pancreatic cancers, colorectal adenocarcinoma, tongue carcinoma, gastric carcinoma, and nasopharyngeal cancers.

Thus, BKB₁R antagonists of formula (1) are useful for treating any one of the conditions, as listed above, wherein BKB₁Rs are induced and over-expressed.

Thus the present invention relates to selective peptidic or peptidoid derivatives BKB₁R antagonists that at least have a good affinity and selectivity for the BKB₁R in comparison with the BKB₁R antagonists developed in the past, are more resistant to in vitro and in vivo enzymatic degradation and present equal to superior pharmacokinetic properties with respect to previously disclosed compounds (Neugebauer et al. vide supra).

For the purpose of the present invention the following terms are defined below.

The term “Ac” is intended to mean acetyl.

The term “AC3” is intended to mean 1-amino-1-cyclopropane-1-carboxylic acid.

The term “AC4” is intended to mean 1-amino-1-cyclobutane-1-carboxylic acid.

The term “AC5” is intended to mean 1-amino-1-cyclopentane-1-carboxylic acid.

The term “AC6” is intended to mean 1-amino-1-cyclohexane-1-carboxylic acid.

The term “Aib” is intended to mean 2-aminoisobutyric acid.

The term “Aic” is intended to mean 2-aminoindane-2-carboxylic acid.

The term “Atc” is intended to mean 2-aminotetraline-2-carboxylic acid.

The term “BK” is intended to mean Bradykinin.

The term “BKB₁R” is intended to mean Bradykinin B1 receptor.

The term “BKB₂R” is intended to mean Bradykinin B2 receptor.

The term “Boc” is intended to mean tert-butyloxy carbonyl.

The term “Cit” is intended to mean Citrulline.

The term “Cha” is intended to mean β-cyclohexyl-alanine.

The term “Chg” is intended to mean α-cyclohexyl-glycine.

The term “Cpa” is intended to mean β-cyclopentyl-alanine.

The term “Cpg” is intended to mean α-cyclopentyl-glycine.

The term “DBU” is intended to mean diazabicyclo[5.4.0]undec-7-ene.

The term “DCC” is intended to mean dicyclohexylcarbodiimide.

The term “DCM” is intended to mean dichloromethane.

The term “DIAD” is intended to mean diisopropyl azodicarbonate.

The term “DIEA” is intended to mean N,N-diisopropylethyl amine.

The term “DME” is intended to mean 1,2-dimethoxyethane.

The term “DMF” is intended to mean N,N-dimethylformamide.

The term “Fmoc” is intended to mean 9-fluorenylmethoxycarbonyl.

The term “HATU” is intended to mean O-(7-azabenzotriazol-1-yl)-1,1,3,3-tetramethyluronium hexafluorophosphate.

The term “Hyp” is intended to mean trans-4-hydroxy-Pro.

The term “IgIb” is intended to mean 2-indanylglycine.

The term “Me” is intended to mean methyl.

The term “Nal” is intended to mean 2-naphthyl-Ala.

The term “O-NBS” is intended to mean ortho-nitrobenzenesulfonyl.

The term “NMP” is intended to mean N-methylpyrrolidinone.

The term “NMO” is intended to mean N-methylmorpholine oxide.

The term “Oic” is intended to mean octahydroindole-2-carboxylic acid.

The term “Phg” is intended to mean phenylglycine.

The term “TBTU” is intended to mean O-(benzotriazol-1-yl)-1,1,3,3-tetramethyluronium tetrafluoroborate.

The term “Thi” is intended to mean β-(2-thienyl)-alanine.

The term “TIPS” is intended to mean triisopropyl silane.

The term “TFA” is intended to mean trifluoroacetic acid.

The term “TPP” is intended to mean triphenylphosphine.

### DETAILED DESCRIPTION OF THE INVENTION

Selected BKB₁R antagonists of the present invention are characterized by an extended hydrophobic side chain at the N-terminal position, this hydrophobic side chain has been found to improve antagonist potency. A variation in the length of the alkyl side chain (6-12 carbon atoms) was done in order to determine the optimal length for antagonist activity. The nature of the side-chain can vary. The hydrophobic extension may serve as a linker to attach other molecules to the bradykinin antagonist where R is —(CH₂)ₘ—CO—C₄H₆—OCH₃, and m is 2 or 3. BKB₁R antagonists of the present invention may be illustrated by the following:

| R | (Aaa⁰ | Arg¹ | Aaa² | Aaa³ | Aaa⁴ | Aaa⁵ | Ser⁶ | D-βNal⁷ | Ile⁸ | OH)₁ |
|---|---|---|---|---|---|---|---|---|---|---|
| n-C₅H₁₁CO | Orn | Arg | Oic | Pro | Gly | α(Me)Phe | Ser | D-βNal | Ile | OH |
| n-C₇H₁₅CO | Orn | Arg | Oic | Pro | Gly | α(Me)Phe | Ser | D-βNal | Ile | OH |
| n-C₉H₁₉CO | Orn | Arg | Oic | Pro | Gly | α(Me)Phe | Ser | D-βNal | Ile | OH |
| n-C₁₁H₂₃CO | Orn | Arg | Oic | Pro | Gly | α(Me)Phe | Ser | D-βNal | Ile | OH |
| pMeO—C₆H₄—COC₄H₈CO | Orn | Arg | Oic | Pro | Gly | α(Me)Phe | Ser | D-βNal | Ile | OH |
| pMeO—C₆H₄—COC₆H₁₂CO | Orn | Arg | Oic | Pro | Gly | α(Me)Phe | Ser | D-βNal | Ile | OH |
| pMeO—C₆H₄—CO—C₄H₈CO | Lys | Arg | Pro | Pro | Gly | Phe | Ser | D-βNal | Ile | OH |
| pMeO—C₆H₄—COC₆H₁₂CO | Lys | Arg | Pro | Pro | Gly | Phe | Ser | D-βNal | Ile | OH |
| n-C₆H₁₃ | Orn | Arg | Oic | Pro | Gly | α(Me)Phe | Ser | D-βNal | Ile | OH |
| n-C₈H₁₇ | Orn | Arg | Oic | Pro | Gly | α(Me)Phe | Ser | D-βNal | Ile | OH |
| n-C₁₀H₂₁ | Orn | Arg | Oic | Pro | Gly | α(Me)Phe | Ser | D-βNal | Ile | OH |
| n-C₁₂H₂₅ | Orn | Arg | Oic | Pro | Gly | α(Me)Phe | Ser | D-βNal | Ile | OH |

Representative bradykinin peptidic or peptidoid antagonists of the present invention may be illustrated by the following:

| (R | Aaa⁰ | Arg¹ | Aaa² | Aaa³ | Aaa⁴ | Aaa⁵ | Ser⁶ | D-βNal⁷ | Ile⁸ | OH)₁ |
|---|---|---|---|---|---|---|---|---|---|---|
| CH₃ | Orn | Arg | Oic | Pro | Gly | α(Me)Phe | Ser | D-βNal | Ile | OH |
| C₂H₅ | Orn | Arg | Oic | Pro | Gly | α(Me)Phe | Ser | D-βNal | Ile | OH |
| n-C₃H₇ | Orn | Arg | Oic | Pro | Gly | α(Me)Phe | Ser | D-βNal | Ile | OH |
| n-C₄H₉ | Orn | Arg | Oic | Pro | Gly | α(Me)Phe | Ser | D-βNal | Ile | OH |
| Ac | Orn | Arg | Oic | Hyp | Gly | α(Me)Phe | Ser | D-βNal | Ile | OH |
| Ac | Orn | Arg | Oic | Pro | Gly | D-α(Me)Phe | Ser | D-βNal | Ile | OH |
| Ac | Orn | Arg | Oic | Pro | Gly | D-Phe | Ser | D-βNal | Ile | OH |
| Ac | Orn | Arg | Oic | Pro | Gly | Cha | Ser | D-βNal | Ile | OH |
| Ac | Orn | Arg | Oic | Pro | Gly | Cpa | Ser | D-βNal | Ile | OH |
| Ac | Orn | Arg | Oic | Pro | Gly | Phg | Ser | D-βNal | Ile | OH |
| Ac | Orn | Arg | Oic | Pro | Gly | Atc | Ser | D-βNal | Ile | OH |
| Ac | Orn | Arg | Oic | Pro | Gly | Thi | Ser | D-βNal | Ile | OH |
| Ac | Orn | Arg | Oic | Pro | Gly | Iglb | Ser | D-βNal | Ile | OH |
| Ac | Orn | Arg | Oic | Pro | Gly | Aic | Ser | D-βNal | Ile | OH |
| Ac | Orn | Arg | Oic | Pro | Gly | Chg | Ser | D-βNal | Ile | OH |
| Ac | Orn | Arg | Oic | Pro | Gly | Cpg | Ser | D-βNal | Ile | OH |
| Ac | Orn | Arg | Oic | Pro | Gly | Aib | Ser | D-βNal | Ile | OH |
| Ac | Orn | Arg | Oic | Pro | Gly | AC6 | Ser | D-βNal | Ile | OH |
| Ac | Orn | Arg | Oic | Pro | Gly | AC5 | Ser | D-βNal | Ile | OH |
| Ac | Orn | Arg | Oic | Pro | Gly | AC4 | Ser | D-βNal | Ile | OH |
| Ac | Orn | Arg | Oic | Pro | Gly | AC3 | Ser | D-βNal | Ile | OH |
| Ac | Lys | Arg | Pro | Pro | Gly | D-Phe | Ser | D-βNal | Ile | OH |
| Ac | Lys | Arg | Pro | Pro | NH—CH₂—CH₂ | Phe | Ser | D-βNal | Ile | OH |
| Ac | Orn | Arg | Oic | Pro | NH—CH₂—CH₂ | Phe | Ser | D-βNal | Ile | OH |
| Ac | Orn | Arg | NH—CH₂—C₆H₄—CH₂—CO | | | α(Me)Phe | Ser | D-βNal | Ile | OH |
| Ac | Orn | Arg | NH—C₆H₄—CH₂—CO | | | α(Me)Phe | Ser | D-βNal | Ile | OH |
| Ac | Orn | Arg | NH—CH₂-biphenyl-CO | | | α(Me)Phe | Ser | D-βNal | Ile | OH |
| Ac | Orn | Arg | NH—(CH₂)₇—CO | | | α(Me)Phe | Ser | D-βNal | Ile | OH |
| Ac | Orn | Arg | NH—(CH₂)₁₀—CO | | | | Ser | D-βNal | Ile | OH |
| Ac | Orn | Arg | amino-ethyl-2,4-dioxo-3,4-dihydro-2H- | | | | Ser | D-βNal | Ile | OH |
| | | | quinazolin-1-yl | | | | | | | |
| Ac | Orn | Arg | piperidin-4-yl-2-oxo-2,3-dihydro- | | | | Ser | D-βNal | Ile | OH |
| | | | benzoimidazol-1-yl | | | | | | | |
| Ac | Orn | Arg | 4-oxo-1-phenyl-1,3,8-triazaspiro [4.5]dec-3- | | | | Ser | D-βNal | Ile | OH |
| | | | yl | | | | | | | |
| Ac | Orn | Arg | 4-oxo-1-cyclohexyl-1,3,8-triazaspiro | | | | Ser | D-βNal | Ile | OH |
| | | | [4.5]dec-3-yl | | | | | | | |
| Ac | Orn | Arg | Oic | Hyp | Gly | Cha | Ser | D-βNal | Ile | OH |
| nC₃H₇CO | Orn | Arg | Oic | Hyp | Gly | Cha | Ser | D-βNal | Ile | OH |
| Ac | Orn | Arg | Oic | Hyp | Gly | Thi | Ser | D-βNal | Ile | OH |
| nC₃H₇CO | Orn | Arg | Oic | Hyp | Gly | Thi | Ser | D-βNal | Ile | OH |
| Ac | Orn | Arg | Oic | Hyp | Gly | Phg | Ser | D-βNal | Ile | OH |
| nC₃H₇CO | Orn | Arg | Oic | Hyp | Gly | Phg | Ser | D-βNal | Ile | OH |
| Ac | Orn | Arg | Oic | Hyp | Gly | Aic | Ser | D-βNal | Ile | OH |
| nC₃H₇CO | Orn | Arg | Oic | Hyp | Gly | Aic | Ser | D-βNal | Ile | OH |

Representative compounds according to the invention include homo-dimerized BKB₁ receptor antagonist as described previously and further defined by the formula:

| R | (Aaa⁰ | Arg¹ | Aaa² | Aaa³ | Aaa⁴ | Aaa⁵ | Ser⁶ | D-βNal⁷ | Ile⁸ | OH)₂ |
|---|---|---|---|---|---|---|---|---|---|---|
| [C₂H₄—CO]₂ | (Orn | Arg | Oic | Pro | Gly | α(Me)Phe | Ser | D-βNal | Ile | OH)₂ |
| [n-C₃H₆—CO]₂ | (Orn | Arg | Oic | Pro | Gly | α(Me)Phe | Ser | D-βNal | Ile | OH)₂ |
| [n-C₄H₈—CO]₂ | (Orn | Arg | Oic | Pro | Gly | α(Me)Phe | Ser | D-βNal | Ile | OH)₂ |
| [n-C₅H₁₀—CO]₂ | (Orn | Arg | Oic | Pro | Gly | α(Me)Phe | Ser | D-βNal | Ile | OH)₂ |
| [C₂H₄—CO]₂ | (Lys | Arg | Pro | Pro | Gly | Phe | Ser | D-βNal | Ile | OH)₂ |
| [n-C₃H₆—CO]₂ | (Lys | Arg | Pro | Pro | Gly | Phe | Ser | D-βNal | Ile | OH)₂ |
| [n-C₄H₈—CO]₂ | (Lys | Arg | Pro | Pro | Gly | Phe | Ser | D-βNal | Ile | OH)₂ |
| [n-C₅H₁₀—CO]₂ | (Lys | Arg | Pro | Pro | Gly | Phe | Ser | D-βNal | Ile | OH)₂ |
| [n-C₃H₆]₂ | (Orn | Arg | Oic | Pro | Gly | α(Me)Phe | Ser | D-βNal | Ile | OH)₂ |
| [n-C₄H₈]₂ | (Orn | Arg | Oic | Pro | Gly | α(Me)Phe | Ser | D-βNal | Ile | OH)₂ |
| [n-C₅H₁₀]₂ | (Orn | Arg | Oic | Pro | Gly | α(Me)Phe | Ser | D-βNal | Ile | OH)₂ |
| [n-C₆H₁₂]₂ | (Orn | Arg | Oic | Pro | Gly | α(Me)Phe | Ser | D-βNal | Ile | OH)₂ |
| [n-C₃H₆]₂ | (Lys | Arg | Pro | Pro | Gly | Phe | Ser | D-βNal | Ile | OH)₂ |
| [n-C₄H₈]₂ | (Lys | Arg | Pro | Pro | Gly | Phe | Ser | D-βNal | Ile | OH)₂ |
| [n-C₅H₁₀]₂ | (Lys | Arg | Pro | Pro | Gly | Phe | Ser | D-βNal | Ile | OH)₂ |
| [n-C₆H₁₂]₂ | (Lys | Arg | Pro | Pro | Gly | Phe | Ser | D-βNal | Ile | OH)₂ |

In the above compounds, R represents either a terminal group or in case of the dimer, R represents a spacer between the monomeric units.

As used herein, the term “aliphatic” means alkyl (C₁-C₁₂), alkenyl (C₂-C₁₂), or alkynyl (C₂-C₁₂).

As used herein, the term “aromatic” means mono or bi-cyclic six-membered rings, and are substituted with alkyl (C₁-C₄), halo, cyano, nitro, amino, hydroxyl, alkoxy groups and the like.

As used herein, the term “heterocycloalkyl” means a cycloalkyl where one to three carbon atoms is replaced with a heteroatom, such as O, NR (R═H, alkyl, aromatic, cycloalkyl) and the like. This term includes residues in which one or more rings is optionally substituted with up to one substituent.

As used herein, the term “alicyclic” means optionally substituted cycloalkyl (C₄-C₁₂), optionally containing 1-3 double bonds.

As used herein, the term “pA2” means: -log10 of the molar concentration of antagonist that reduces the effect of a double concentration of agonist to that of a single one.

As used herein, the term “substituted” means alkyl, alkenyl, cycloalkyl, aryl, heteroaryl, or heterocycloalkyl, wherein hydrogen atoms are replaced by halogen, hydroxyl, carboxy, carboalkoxy, carboamido, cyano, carbonyl, alkylamino, dialkylamino, acylamino, aminosulfonyl, phenyl, benzyl, trityl, phenoxy, amidino, guanidine, ureido, or benzyloxy.

As used herein, the term “pharmaceutically acceptable salt” refers to salts prepared from pharmaceutical acceptable acids or bases including inorganic acids and bases or organic acids and bases. When the compounds of the present invention contain a basic side chain, salts may be prepared from pharmaceutical acceptable acids including inorganic or organic acids. Suitable pharmaceutically acceptable acid addition salts for the compound of the present invention include salts of acetic acid, trifluoroacetic acid, benzenesulfonic acid, benzoic acid, camphorsulphonic acid, citric acid, ethensulfonic acid, fumaric acid, gluconic acid, glutamic acid, hydrobromic acid, hydrochloric acid, isethionic acid, lactic acid, maleic acid, malic acid, mandelic acid, methanesulfonic acid, mucic acid, nitric acid, pamoic acid, pantothenic acid, phosphoric acid, succinic acid, sulfuric acid, tartaric acid, p-toluenesulfonic acid, and the like. Suitable pharmaceutically acceptable base addition salts for the compounds of the present invention include metallic salts made from aluminium, calcium, lithium, magnesium, potassium, sodium, and zinc or organic salts made from lysine, N,N′-dibenzylethylenediamine, chloroprocaine, choline, diethanolamine, ethylenediamine, meglumine (N-methylglucamine) or procaine.

As used herein, the term “anti-allergy agent” refers to an agent or a compound useful to treat allergy.

As used herein, the term “anti-angiogenic agent” refers to an agent or a compound useful to prevent or block angiogenesis.

As used herein, the term “anti-cancer agent” refers to an agent or a compound useful to treat cancer.

As used herein, the term “an anti-inflammatory agent” refers to an agent or a compound having anti-inflammatory activity.

The present invention will be more readily understood by referring to the following examples which are given to illustrate the invention rather than to limit its scope.

### EXAMPLE I

### Peptide Synthesis

Synthesis of the BKB₁R antagonists of the present invention by solid phase peptide synthesis (SPPS) may be carried out manually (see Stewart & Young and K. Wisniewski) or by use of the Applied Bioscience 430A for Boc-amino acids or by use of Pioneer™ continuous flow peptide synthesis system for Fmoc-amino acids. On-resin formation of the GlyΨ[CH₂]Aaa type reduced peptide bond by Mitsunobu alkylation is described in PEPTIDES 2002-Proceedings of the 27^{th} European Peptide Symposium, Sorrento, 2002 by K. Wisniewski (pp. 322-3). Solid phase peptide synthesis involves use of standard procedures, defined as follows:

General Method Involving Boc-Strategy

Procedure A

DCC Coupling Reaction

A 4-fold excess of Boc-amino acids over resin substitution rate is used in the Applied Bioscience 430A synthesizer. Boc-amino acids are activated for coupling with an equimolar amount of DCC and 2 equivalents of DIEA. The solvent may be DCM, DMF, or NMP. The resin is washed with the same solvent before and after coupling. Completeness of coupling is determined with a Kaiser test.

Procedure B

TFA Deprotection and Neutralization

The deprotection reagent is 40% TFA in DCM, containing 1 mg/mL N-acetyl-LD-tryptophan. It is used for 30 min, following a prewash. The neutralization reagent is 20% DIEA in DCM.

Procedure C

N-Terminal Acylation

A 5-fold excess of acyl chlorides and 10-fold excess of DIEA over peptide-resin are used in DCM for 30 min. The resin is washed with the same solvent after completion of the reaction.

Procedure D

HF Cleavage

A batch of 0.5 mmole of peptide-resin is mixed with 1.0 mL anisole and chilled in the reaction vessel (resistant to HF) to −78 ° C., and 10 ml of anhydrous HF is distilled into the vessel under vacuum. The mixture is stirred at 0° C. for 1 h, and the HF is evaporated first under a nitrogen flow, then under vacuum. The peptide and resin mixture is washed three times with dry ether, and the peptide is extracted into 50% acetic acid. The peptide solution is concentrated under vacuum, diluted in water, and lyophilized.

Procedure E

Purification

Preparative medium pressure chromatography may be carried out on a reversed phase C18 silica column in a gradient of 0.1% TFA in water to 0.05% TFA in acetonitrile. Eluted peptide is detected by UV at 254 nm. Analytical HPLC may be carried out in the same system to identified pure fractions.

Procedure F

Characterization

Final products are identified by analytical HPLC and by mass spectroscopy (Table 1). MALDI spectra are recorded on a Tofspec 2E (micromass, UK) in mode reflectron.

General Method Involving Fmoc-Strategy

The approach is used in the preparation of peptides having Orn and α(Me)Phe residues. Synthesis may be carried out by use of Pioneer™ continuous flow peptide synthesis system.

Procedure G:

The resin is placed in the column and a 2 to 4-fold excess of Fmoc-protected amino acids over resin substitution rate is placed in the sampler tray. Synthesis is performed using amine free DMF. All solutions needed for the solid phase continuous flow synthesis are prepared and loaded in the synthesizer. The synthesis protocol is prepared, loaded into the synthesizer, and run in normal or extended cycle mode. Fmoc deprotection is performed in 20% piperidine in DMF and monitored through UV detector at 364 nm. Fmoc-protected amino acids are activated for coupling with an equimolar amount of HATU or TBTU, and 2 equivalents of DIEA.

Procedure H

N-terminal Caping (Acetylation)

This step is optional and can be included in the synthesis protocol. The acetylation reagents are 5% acetic anhydride and 6% 2,4-lutidine in DMF. The resin is washed with the same solvent and isopropanol after completion of the reaction. The resin is removed from the column synthesizer and dried under vacuum 12 hours.

Procedure I

TFA Cleavage

The cleavage solution, TFA:water:TIPS (95%:2.5%:2.5%), is mixed with peptide-resin, and stirred at room temperature for 2 h. The resin is filtrated and the peptide is precipitated in dry ether. The suspension is centrifuged. The ether solution is decanted and the precipitated peptide is dissolved in water and lyophilized. The peptide is purified and characterized as described in procedures E and F.

### EXAMPLE II

### Formation of the GlyΨ[CH₂]Aaa Type Reduced Peptide Bond

Procedure J

The peptide chain is assembled by Fmoc strategy. O-NBS group is introduced after Fmoc deprotection at the site of the intended peptide reduced bond by adding 1.5-fold excess of O-NBS-chloride to peptide-resin swelled in 2,4,6-collidine. The mixture is stirred at room temperature for 12 h, and completeness of protection is determined with a Kaiser test. 0.1 mmol of O-NBS-Aaaₙ-resin (0.2-0.6 mmol/g) is suspended in 1 mL of DME, and 1 mmol of Fmoc-Gly-ol is added to the suspension. The DIAD/TPP complex is preformed at 0° C. by mixing 1 mL of 1M TPP in DME and 1 mL of 1M DIAD in DME. The mixture is stirred an additional 5 min, and subsequently added to the resin suspension. The suspension is shaken overnight. To assess the completeness of the reaction, a small aliquot of resin is cleaved with 95% TFA/H₂O and the sample is analyzed by analytical HPLC, and compared to cleaved O-NBS-Aaaₙ peptide. After the desired peptide is assembled, the resin is treated with 10 equivalent of 1 M solution of mercaptoethanol/DBU in DMF for 1 h, and washed thoroughly with DMF and DCM. The peptide is then cleaved with an appropriate TFA cocktail, see procedure I. The peptide is purified and characterized as described in procedures E and F.

### EXAMPLE III

### Formation of Synthesis of N-Terminal Alkylated Analogues

The peptide chain is assembled by Fmoc strategy. O-NBS group is introduced after Fmoc deprotection at the N-terminal position as described in procedure J followed by 0.1 mmol of O-NBS-Aaaₙ-resin (0.2-0.6 mmol/g) is suspended in 1 mL of DME, and 1 mmol of appropriate alcohol is added to the suspension. Mistunobu alkylation with DIAD/TPP and deprotection of the O-NBS group are performed as described in procedure J. After the desired peptide is assembled, the resin is treated with 10 equivalent of 1 M solution of mercaptoethanol/DBU in DMF for 1 h, and washed thoroughly with DMF and DCM. The peptide is then cleaved with an appropriate TFA cocktail, see procedure I. The peptide is purified and characterized as described in procedures E and F.

### EXAMPLE IV

### Synthesis of Homo-Dimers

The peptide chain is assembled by Boc or Fmoc strategy. Diacyl spacer is introduced after Boc or FrMoc deprotection at the N-terminal position by treating the peptide-resin with 0.6 equivalent of the appropriate diacid chloride and 10 equivalents of DIEA in DCM. The mixture is stirred at room temperature for 30 min, and completeness of reaction is determined with a Kaiser test. The peptide-resin is washed thoroughly with DCM. The peptide is then cleaved with an appropriate TFA cocktail, see procedure I. The peptide is purified and characterized as described in procedures E and F.

### EXAMPLE V

### In Vitro Bioassays to Assess the Selectivity to, and Potency Against, the Inducible BKB₁R Subtype (Isolated Preparations in Organ Baths and Cultured Cell Binding)

Selected antagonists were tested for activities in three isolated organs: (1) the rabbit aorta (rbA), (2) the human umbilical vein (hUV) and (3) the rabbit jugular vein (rbJV).

All details regarding the procurements of human umbilical cords and rabbit vessels, as well as the procedures for preparing the isolated organs and the experimental protocols are described in these respective publications: rbA (Rioux et al. 1973, Can J Physiol Pharmacol, 51: 114-121); hUV (Gobeil et al. 1996, Br J Pharmacol, 118: 289-294), and rbJV (Gaudreau et al. 1981, Can J Physiol Pharmacol, 59: 371-379).

The rabbit aorta without endothelium (which contains only the BKB₁R) was used to determine the antagonistic activities of each compound.

hUV that contains BKB₁ and BKB₂ receptors was treated with HOE 140 (Icatiban™; Jerini Inc.), a potent, selective and specific BKB₂R antagonist, to eliminate any activation (action/response) of the constitutive BKB₂R subtype in experiments intended to measure the antagonistic activity of each compound in BKB₁ receptor challenged with either Lys-desArg⁹BK or DesArg⁹BK, two selective BKB₁R agonists.

The rabbit jugular vein (a pure BKB₂ receptor system) was used to exclude any action of the new compounds on the BKB₂ receptor and thus establish their selectivity toward the BKB₁R. All tissues were treated with captopril (1 μM) to prevent the degradation of the peptidic agonists.

Repeated applications of a single and double concentration of the natural BK (on rbJV,) as a dual agonist to both receptor subtypes, or of Lys-desArg⁹BK (rbA and hUV), a selective BKB₁R agonist, were made in the absence and in presence of the various peptides and peptidoids analogs synthesized herein to evaluate their apparent affinities as antagonists, in terms of pA2 (-log10 of the molar concentration of antagonist that reduces the effect of a double concentration of agonist to that of a single one), (Schild 1947, Br J Pharmacol, 2: 189-206). The antagonists were applied 10 min before measuring the myotropic effects of either BK or Lys-desArg⁹BK. All compounds tested as potential antagonists were initially applied to these three tissues from two species at the concentration of 10 μM to measure their “potential agonistic activities, (αE)” in comparison with BK (in the BKB₂R preparations) or Lys-desArg⁹BK (in the BKB₁R preparations). Compounds of the present invention exhibit pA2 value ranging from 6 to 9.5, when tested in models for in vitro BKB₁R isolated animal (rabbit) and human tissue bioassays.

### EXAMPLE VI

### In Vitro Binding Studies to Assess the Potency of Molecules at the Inducible BKB1 and Constitutive BKB₂ Receptor Subtypes (Cultured Cells)

Radioligand binding assays on native human BKB₁ and BKB₂ receptors were performed as previously described with modifications (Faussner et al., 1998, J Biol Chem, 273: 2617-2623; Gobeil et al., 2003, J Biol Chem, 278: 38875-38883). Briefly, IMR-90 cells (human lung fibroblasts) were seeded into 24-well plates (50000 cells/well, 500 μl/well) and allowed to reach 90% confluency before beginning experiments. For binding assays at BKB₁Rs, cells were exposed to IL-1β (0.5 ng/ml) overnight at 37° C. prior to experiments in order to increase BKB₁R expression. Cells were then washed twice with ice-cold binding buffer consisting of PBS 1× containing CaCl₂ (0.13 g/l), MgCl₂ (0.1 g/l) 0.1% bovine serum albumin (fatty acid free) and supplemented with protease inhibitors 10 μM captopril, 10 μM thiorphan and 10 μM mergetpa. For saturation or displacement curves, IMR-90 cells were incubated at room temperature (23° C.) for 60 min in the above-mentioned buffer in the presence of various concentrations of [³H]BK or [³H]LysdesArg⁹BK ranging from 0.1-20 nM. Kd and Bmax values were determined from Scatchard analysis. Displacement binding was performed using 0.5 nM of radioligand and unlabeled ligand in the range of 0.01-10000 nM. Ki values were calculated from the IC₅₀ value (concentration of unlabeled ligand causing 50% displacement of specific binding) using the Cheng-Prusoff approximation (Cheng and Prusoff, 1973, Biochem Pharmacol, 22: 3099-3108). Non specific binding was determined in the presence of 5 μM of the appropriate unlabeled ligand. After the incubation period, cells were washed twice with ice-cold binding buffer, lysed with 0.1 N NaOH (200 μl/well), and transferred into scintillation vials. Radioactivity in the samples was measured in a beta counter after addition of a 20-fold volume of scintillation cocktail (4 ml/vial). In parallel, cells from untreated wells within the same plate were treated with trypsin-EDTA and counted with a hemacytometer for cell count normalization. Specific binding was expressed in DPM/well or fmol/well). Data were analyzed using GraphPad computer software (PRISM software, GraphPad, CA).

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

### List of International Patents (Publication Date) and References (Alphabetical Order) Cited in the Present Patent Application

WO97/09346

WO97/25315

WO00/75107

WO01/05783

WO02/099388

WO03/106428

WO03/066577

IUPAC-IUB Commission on Biochemical Nomenclature: Symbols for amino acids derivatives and peptides 1972, Biochem J 126, 773-780.

Cheng and Prusoff, 1973, Biochem Pharmacol 22, 3099-3108.

Couture et al. 2001, Eur J Pharmacol 429, 161-176.

Davis and Perkins, 1994, Neuropharmacol 33, 127-133.

Dray and Perkins, 1993, Trends Neurosci 16, 99-104.

Emanueli et al., 2002, Circ 105, 360-366.

Farmer S. G., 1997, The Kinin System, Academic Press, San Diego, 349 P.

Faussner et al., 1998, J Biol Chem 273, 2617-2623.

Gabra and Sirois, 2003, Peptides 24, 1131-1139.

Gaudreau et al. 1981, Can J Physiol Pharmacol 59, 371-379.

Gobeil et al. 1996, Br J Pharmacol 118, 289-294.

Gobeil et al., 2003, J Biol Chem 278, 38875-38883.

Ishihara et al., 2002, Int Immunopharmacol 2, 499-509.

Marceau et al. 1998, Pharmacol Rev 50, 357-386.

Neugebauer et al. 2002, Can J Physiol Pharmacol 80, 287-292.

Ozturk et al. 2001, Curr Pharma Des 7, 135-161.

Perron et al., 1999, Eur J Pharmacol 376, 83-89.

Regoli and Barabe, 1980, Pharmacol Rev 32, 1-46.

Regoli et al. 1977, Can J Physiol Pharmacol 55, 855-867.

Resende et al., 1998, Braz J Med Biol Res 31, 1229-1235.

Rioux et al. 1973, Can J Physiol Pharmacol 51, 114-121.

Schild 1947, Br J Pharmacol 2, 189-206.

Simard et al., 2002, Can J Physiol Pharmacol 80, 1203-1207

Stewart and Young 1984, Solid Phase Synthesis, Sec. Ed., Pierce Chemical Co.

Skidgel, 1992, J Cardiovasc Pharmacol 20, S4-9.

Spillmann et al., 2002, Int Immunopharmacol, 2, 1823-1832.

Wisniewski 2002, PEPTIDES Proceedings of the 27^{th} European Peptide Symposium, Sorrento, 322-323.

Zuccollo et al. 1996, Can J Physiol Pharmacol 74, 586-589.

1. A compound of the formula (1)
R-(Aaa⁰-Arg¹-Aaa²-Aaa³-Aaa⁴-Aaa⁵-Ser⁶-D-βNal⁷-Aaa⁸-OH)ₓ (1);
R is an acetyl group, or a hydrophobic extension;
Aaa⁰ is Orn, Lys, a basic amino acid, or a salt thereof;
Aaa² is Oic, Pro, a Pro mimic amino acid, or a Pro mimic derivative;
Aaa³ is Pro, or a Pro mimic amino acid, or a Pro mimic derivative;
Aaa⁴ is Gly, or H₂N—(CH₂)₂, or Aib;
Aaa⁵ is α(Me)Phe, Phe, D-α(Me)Phe, D-Phe, Cha, Cpa, Phg, Atc, Thi, Iglb, Aic, Chg, Cpg, Aib, AC6, AC5, AC4, AC3;
Aaa⁸ is Ile, or Leu, or Nle; and
x is 1 or 2,
or,
-Aaa²-Aaa³-Aaa⁴- together form a group selected from aliphatic, aromatic-aliphatic heterocyclic or alicyclic group,
or,
-Aaa²-Aaa³-Aaa⁴-Aaa⁵- together form a group selected from aliphatic, heterocyclic or alicyclic group.

2. The compound of claim 1, wherein Aaa⁵ is selected from the group consisting of α(Me)Phe, Cha, Thi, Phg, and Aic.

3. The compound of claim 1, wherein the hydrophobic extension is an aliphatic or an aromatic-aliphatic acylating group.

4. The compound of claim 1, wherein the basic amino acid Aaa^{0 is Arg or Cit.}

5. The compound of claim 1, wherein the in Aaa² is Hyp or α(Me)Pro.

6. The compound of claim 1, wherein the Pro mimic amino acid in Aaa³ is Hyp, Oic or α(Me)Pro.

7. The compound of claim 1, wherein said compound is in free base form or in salt form with an acid or a base.

8. A compound selected from the group consisting of:
i) n-C₅H₁₁—CO-Orn-Arg-Oic-Pro-Glyα(Me)Phe-Ser-D-βNal-Ile-OH;
ii) n-C₇H₁₅—CO-Orn-Arg-Oic-Pro-Glyα(Me)Phe-Ser-D-βNal-Ile-OH;
iii) n-C₉H₁₉—CO-Orn-Arg-Oic-Pro-Glyα(Me)Phe-Ser-D-βNal-Ile-OH;
iv) n-C₁₁H₂₃—CO-Orn-Arg-Oic-Pro-Glyα(Me)Phe-Ser-D-βNal-Ile-OH;
v) pMeO—C₆H₄—CO—C₄H₈CO-Orn-Arg-Pro-Gly-α(Me)Phe-Ser-D-βNal-Ile-OH;
vi) pMeO—C₆H₄—CO—C₆H₁₂CO-Orn-Arg-Pro-Gly-α(Me)Phe-Ser-D-βNal-Ile-OH;
vii) pMeO—C₆H₄—CO—C₆H₈CO-Orn-Arg-Pro-Gly-α(Me)Phe-Ser-D-βNal-Ile-OH;
viii) pMeO—C₆H₄—CO—CH₆H₁₂—CO-Lys-Arg-Pro-Pro-Gly-Phe-Ser-D-βNal-Ile-OH;
ix) CH₃-Orn-Arg-Oic-Pro-Gly-α(me)Phe-Ser-D-βNal-Ile-OH;
x) C₂H₅-Orn-Arg-Oic-Pro-Gly-α(Me)Phe-Ser-D-βNal-Ile-OH;
xi) n-C₃H₇-Orn-Arg-Oic-Pro-Gly-α(Me)Phe-Ser-D-βNal-Ile-OH;
xii) n-C₄H₉-Orn-Arg-Oic-Pro-Gly-α(Me)Phe-Ser-D-βNal-Ile-OH;
xiii) n-C₆H₁₃-Orn-Arg-Oic-Pro-Gly-α(Me)Phe-Ser-D-βNal-Ile-OH;
xiv) n-C₈H₁₇-Orn-Arg-Oic-Pro-Gly-α(Me)Phe-Ser-D-βNal-Ile-OH;
xv) n-C₁₉H₂₁-Orn-Arg-Oic-Pro-Gly-α(Me)Phe-Ser-D-βNal-Ile-OH;
xvi) n-C₁₂H₂₅-Orn-Arg-Oic-Pro-Gly-α(Me)Phe-Ser-D-βNal-Ile-OH;
xvii) Ac-Orn-Arg-Oic-Hyp-Gly-α(Me)Phe-Ser-D-βNal-Ile-OH;
xviii) Ac-Orn-Arg-Oic-Pro-Gly-D-α(Me)Phe-Ser-D-βNal-Ile-OH;
xix) Ac-Orn-Arg-Oic-Pro-Gly-D-Phe-Ser-D-βNal-Ile-OH;
xx) Ac-Orn-Arg-Oic-Pro-Gly-Cha-Ser-D-βNal-Ile-OH;
xxi) Ac-Orn-Arg-Oic-Pro-Gly-Cpa-Ser-D-βNal-Ile-OH;
xxii) Ac-Orn-Arg-Oic-Pro-Gly-Phg-Ser-D-βNal-Ile-OH;
xxiii) Ac-Orn-Arg-Oic-Pro-Gly-Atc-Ser-D-βNal-Ile-OH;
xxiv) Ac-Orn-Arg-Oic-Pro-Gly-Thi-Ser-D-βNal-Ile-OH;
xxv) Ac-Orn-Arg-Oic-Pro-Gly-Iglb-Ser-D-βNal-Ile-OH;
xxvi) Ac-Orn-Arg-Oic-Pro-Gly-Aic-Ser-D-βNal-Ile-OH;
xxvii) Ac-Orn-Arg-Oic-Pro-Gly-Chg-Ser-D-βNal-Ile-OH;
xxviii) Ac-Orn-Arg-Oic-Pro-Gly-Cpg-Ser-D-βNal-Ile-OH;
xxix) Ac-Orn-Arg-Oic-Pro-Gly-Aib-Ser-D-βNal-Ile-OH;
xxx) Ac-Orn-Arg-Oic-Pro-Gly-AC6-Ser-D-βNal-Ile-OH;
xxxi) Ac-Orn-Arg-Oic-Pro-Gly-AC5-Ser-D-βNal-Ile-OH;
xxxii) Ac-Orn-Arg-Oic-Pro-Gly-AC4-Ser-D-βNal-Ile-OH;
xxxiii) Ac-Orn-Arg-Oic-Pro-Gly-AC3-Ser-D-βNal-Ile-OH;
xxxiv) Ac-Lys-Arg-Pro-Pro-Gly-D-Phe-Ser-DβNal-Ile-OH;
xxxv) Ac-Lys-Arg-Pro-Pro-GlyΨ[CH₂NH]-Phe-Ser-D-βNal-Ile-OH;
xxxvi) Ac-Orn-Arg-Oie-Pro-GlyΨ[CH₂NH]Phe-Ser-DβNal-Ile-OH;
xxxvii) Ac-Orn-Arg-NH-CH₂—C₆H₄—CH₂—CO-α(Me)Phe-Ser-D-βNal-Ile-OH;
xxxviii) Ac-Orn-Arg-NH—CH₆H₄—CH₂—CO-α(Me)Phe-Ser-D-βNal-Ile-OH;
xxxix) Ac-Orn-Arg-NH—CH₂-biphenyl-CO-α(Me)Phe-Ser-D-βNal-Ile-OH;
xl) Ac-Orn-Arg-NH—(CH₂)₂—CO-α(Me)Phe-Ser-D-βNal-Ile-OH;
xli) Ac-Orn-Arg-NH—(CH₂)₁₀—CO-Ser-D-βNal-Ile-OH;
xlii) Ac-Orn-Arg-amino-ethyl-2,4-dioxo-3,4-dihydro-2H-quinazolin-1-yl-Ser-D-βNal-Ile-OH;
xliii) Ac-Orn-Arg-piperidin-4-yl-2-oxo-2,3-dihydro-benzoimidazol-1yl-Ser-D-βNal-Ile-OH;
xliv) Ac-Orn-Arg-NH-4-oxo-1-phenyl-1,3,8-triazspiro[4,5]dec-3-yl-Ser-DβNal-Ile-OH;
xlv) Ac-Orn-Arg-NH-4-oxo-1-cyclohexyl-1,3,8-triazspiro[4,5]dec-3-yl-Ser-DβNal-Ile-OH;
xlvi) Ac-Orn-Arg-Oic-Hyp-Gly-Cha-Ser-D-βNal-Ile-OH;
xlvii) nC₃H₇CO-Orn-Arg-Oic-Hyp-Gly-Cha-Ser-D-βNal-Ile-OH;
xlviii) Ac-Orn-Arg-Oic-Hyp-Gly-Thi-Ser-D-βNal-Ile-OH;
xlix) nC₃H₇CO-Orn-Arg-Oic-Hyp-Gly-Thi-Ser-D-βNal-Ile-OH;
l) Ac-Orn-Arg-Oic-Hyp-Gly-Phg-Ser-DβNal-Ile-OH;
li) nC₃H₇CO-Orn-Arg-Oic-Hyp-Gly-Phg-Ser-D-βNal-Ile-OH;
lii) Ac-Orn-Arg-Oic-Hyp-Gly-Aic-Ser-D-βNal-Ile-OH;
liii) nC₃H₇CO-Orn-Arg-Oic-Hyp-Gly-Aic-Ser-D-βNal-Ile-OH;
liv) [n-C₂H₄—CO-Orn-Arg-Oic-Pro-Gly-α(Me)Phe-Ser-DβNal-Ile-OH]₂;
lv) [n-C₃H₆—CO-Orn-Arg-Oic-Pro-Gly-α(Me)Phe-Ser-D-βNal-Ile-OH]₂;
lvi) [n-C₄H₈—CO-Orn-Arg-Oic-Pro-Gly-α(Me)Phe-Ser-D-βNal-Ile-OH]₂;
lvii) [n-C₅H₁₀—CO-Orn-Arg-Oic-Pro-Gly-α(Me)Phe-Ser-D-βNal-Ile-OH]₂;
lviii) [n-C₂H₄—CO-Lys-Arg-Pro-Pro-Gly-Phe-Ser-D-βNal-Ile-OH]₂;
lix) [n-C₃H₆—CO-Lys-Arg-Pro-Pro-Gly-Phe-Ser-D-βNal-Ile-OH]₂;
lx) [n-C₄H₈—CO-Lys-Arg-Pro-Pro-Gly-Phe-Ser-D-βNal-Ile-OH]₂;
lxi) [n-C₅H₁₀—CO-Lys-Arg-Pro-Pro-Gly-Phe-Ser-D-βNal-Ile-OH]₂;
lxii) [n-C₃H₆-Orn-Arg-Oic-Pro-Gly-α(Me)Phe-Ser-D-βNal-Ile-OH]₂;
lxiii) [n-C₄H₈-Orn-Arg-Oic-Pro-Gly-α(Me)Phe-Ser-D-βNal-Ile-OH]₂;
lxiv) [n-C₅H₁₀-Orn-Arg-Oic-Pro-Gly-α(Me)Phe-Ser-D-βNal-Ile-OH]₂;
lxv) [n-C₆H₁₂-Orn-Arg-Oic-Pro-Gly-α(Me)Phe-Ser-D-βNal-Ile-OH]₂;
lxvi) [n-C₃H₆-Lys-Arg-Pro-Pro-Gly-Phe-Ser-D-βNal-Ile-OH]₂;
lxvii) [n-C₄H₈-Lys-Arg-Pro-Pro-Gly-Phe-Ser-D-βNal-Ile-OH]₂;
lxviii) [n-C₅H₁₀-Lys-Arg-Pro-Pro-Gly-Phe-Ser-D-βNal-Ile-OH]₂; and
lxix) [n-C₆H₁₂-Lys-Arg-Pro-Pro-Gly-Phe-Ser-D-βNal-Ile-OH]₂.

9. A pharmaceutical composition comprising a therapeutically effective amount of a compound as defined in claim 1 and a pharmaceutically acceptable carrier.

10-27. (canceled)

28. A method for treating a BKB1 receptor associated disease comprising the step of administering to a patient in need thereof a compound as defined in claim 1.

29. The method of claim 28 wherein the BKB1 receptor associated disease is selected from a group consisting of diabesity, brain-neurogenic syndromes, vascular syndromes, pulmonary syndromes, respiratory syndromes, renal syndromes, bowel syndromes, skin injury syndromes, arthritis syndromes, dental pain, skin pain, bone pain, cancer pain, perioperative pain, cough, hyperalgesia, vascular, nephropathy, microangiopathy or retinopathy complications of diabesity, chemotherapy-induced neuropathy, asthma, rhinitis, chronic obstructive pulmonary disease, cancer, coronary, cardiovascular diseases, vasospasm, myocardial ischemia, heart failure, hypertension, stroke, and vasculopathies related to microvascular leakage or remodeling.

30-41. (canceled)
